(19) United States
(12) Patent Application Publication
(10) Pub. No.: US 2007/0128611 A1
(43) Pub. Date: Jun. 7, 2007

(54) Negative control probes

(76) Inventors: Charles F. Nelson, San Carlos, CA (US); Bo Curry, Redwood City, CA (US); Nicholas Sampas, San Jose, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/292,588

(22) Filed: Dec. 2, 2005

(51) Int. Cl. C12Q 1/68; G06F 19/00

(52) U.S. Cl. 435/6; 702/20

(57) ABSTRACT

In an embodiment, a method is included for generating a negative control probe sequence for an array including selecting biological probe sequences from the array randomly, generating a plurality of candidate probe sequences by randomly permuting the selected biological probe sequence, and screening the candidate probe sequences for sequence similarity to biologically occurring sequences. An embodiment also includes a computer-readable medium having computer-executable instructions for performing a method for generating a negative control probe sequences. Embodiments can also include an apparatus for generating a negative control sequence for an array, as well as negative control probes, sets of negative control probes and arrays comprising at least one negative control probe.

### BACKGROUND

Chemical arrays have gained prominence in biological research and serve as valuable diagnostic tools in the healthcare industry. A fundamental principle upon which array assays are based is that of specific recognition. Probe molecules affixed to the array can specifically recognize and bind target molecules in a sample, either by sequence-mediated binding affinities, binding affinities based on conformational or topological properties of probe and target molecules, or binding affinities based on spatial distribution of electrical charge on the surfaces of target and probe molecules.

An array generally includes a substrate upon which a regular pattern of features is prepared by various manufacturing processes. The array typically has a grid-like two-dimensional pattern of features. For nucleic acid arrays, each feature of the array contains a large number of oligonucleotides covalently bound to the surface of the feature. These bound oligonucleotides are known as probes. In general, chemically distinct probes are bound to the different features of an array, so that each feature corresponds to a particular known nucleotide sequence.

Once an array has been prepared, the array may be exposed to a sample solution containing target molecules (such as DNA or RNA) labeled with fluorophores, chemiluminescent compounds, or radioactive atoms. The labeled target molecules then hybridize to the complementary probe molecules on the surface of the array. Targets, such as labeled DNA molecules that are not complementary to any of the probes bound to array surface do not hybridize as readily and tend to remain in solution. The sample solution is then rinsed from the surface of the array, washing away any unbound labeled molecules. Finally, the bound labeled molecules are detected via optical or radiometric scanning.

Scanning of an array by an optical scanning device or radiometric scanning device generally produces a scanned image comprising a plurality of pixels corresponding to features on the array, with each pixel having a corresponding signal intensity. Typically, an array-data-processing program then manipulates these signal intensities and produces experimental or diagnostic results.

Array systems generally have at least some amount of background signals (noise) that is detected. This background noise can be caused by various factors including non-specific binding between probe molecules and labeled target molecules as well as base-specific fluorescence contamination introduced during array manufacture. Accordingly, the measured signal intensity of features on the array generally is corrected for measured background noise in the array system.

One approach to measuring background noise is to place negative control probes on the array that contain internal structures, such as hairpins, that inhibit binding with target sample components. These types of negative control probes (“structured negative controls”) can be good estimators of spatially distributed background noise but have limited ability to measure sequence-dependent background noise. Such probes tend to underestimate both the median background noise and the constant noise level due to variations of the background noise level. Another approach to measuring background noise is to use those biological features on the array exhibiting the weakest signaling levels to estimate sequence-dependent backgrounds. However, because weakest signaling features frequently include some component of true signal, this approach can overestimate both the median background noise and the constant background noise level.

### SUMMARY

In one aspect, the invention provides a method for generating a negative control probe sequence for an array. The method comprises selecting biological probe sequences randomly, generating a plurality of candidate probe sequences by randomly permuting the selected biological probe sequence, and screening the candidate probe sequences for sequence similarity to biologically occurring sequences. In certain aspects, the candidate probes are variable in sequence and matched in base content and melting temperature to other probes on the array but are not substantially complementary to nucleic acids expected to be in a sample under investigation, i.e., the probes do not hybridize to nucleic acids expected to be in a sample under investigation under stringent conditions.

One aspect is a method for generating a negative control probe sequence for an array. The method comprising randomly generating a plurality of candidate negative control probes, screening the candidate negative control probes for sequence similarity to biologically occurring sequences, and screening the candidate negative control probes for one or more of base composition properties, primary structural features, secondary structural features, or thermodynamic characteristics.

Another aspect is a computer-readable medium having computer-executable instructions for performing a method for generating a negative control probe sequence.

Another aspect is an apparatus for generating or designing a negative control sequence for an array. The apparatus comprises a memory store, and a programmable circuit in electrical communication with the memory store. The programmable circuit can be programmed to select biological probe sequences randomly, generate a plurality of candidate probe sequences by randomly permuting the selected biological probe sequence, and screen the candidate probe sequences for sequence similarity to biologically occurring sequences. In another aspect, the probe sequences are screened for one or more of base composition properties, primary structural features, secondary structural features, or thermodynamic characteristics.

Another aspect is an isolated nucleotide sequence comprising or consisting of the sequence:

SEQ ID NO. 1:
5′-TATCCTACTATACGTATCACATAGCGTTCCGTATGTGGCCGGGATAGACCTAGCTTAAGC-
3′
SEQ ID NO. 2:
5′-
ACTCAAATACGGCCGATCTCCGTAGTAAGGCATCCAACCTGCGATACTAGCCACTTCCCG-3′
SEQ ID NO. 3:
5′-
ACAGCCAACTAATCCGGGATACCGCCGTTATTCGACTAATCCCGGGACGTCAAGTTCCAC-3′
SEQ ID NO. 4:
5′-
CCGCGCGGCATGAAGTATGCAGCGCTCGAGCCTAGTCATTCGTAAGCGATATGTTTAGTG-3′
SEQ ID NO. 5:
5′-CGTTTCTACGCGTACGCCTTTATGTCGAGGCAACGCCTCGGTGTACTCCTACGGGTTTTG-
3′
SEQ ID NO. 6:
5′-
ACTGATTGCCGTGTATTAGCCGGTCGGTAACTCGGTTCCGCTACTAGCGCGCCAGATTTC-3′
SEQ ID NO. 7:
5′-
CTAACGGGTCCAAGACGCGCAACATTATGTAGCGTACTAGGACCCTAACTGCGACTATCC-3′
SEQ ID NO. 8:
5′-
CCATAAGGCGGACCCAGATCGATTGACGGGTGGCTAGATATGTCGTGCTTAGTTCCCAAA-3′
SEQ ID NO. 9:
5′-
AGTATGTGTAGCGAGGAGCTAGTCGTCGGTGCACAATCGGCCTAGAATTAGTTGCCTCGA-3′

### BRIEF DESCRIPTION OF THE DRAWINGS

Embodiments may be more completely understood in connection with the following drawings, in which:

FIG. 1 illustrates a schematic diagram of a system for manufacturing arrays.

FIG. 2 illustrates an example of a general purpose computing system.

FIG. 3 shows operations performed in some embodiments.

FIG. 4 shows operations of similarity screening performed in some embodiments.

### DETAILED DESCRIPTION

Before describing the present invention in detail, it is to be understood that this invention is not limited to specific compositions, method steps, or equipment, as such may vary. It is also to be understood that the terminology used herein is for the purpose of describing particular embodiments only, and is not intended to be limiting. Methods recited herein may be carried out in any order of the recited events that is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

Unless defined otherwise below, all technical and scientific terms used herein have the same meaning as commonly understood by one of ordinary skill in the art to which this invention belongs. Still, certain elements are defined herein for the sake of clarity.

All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It must be noted that, as used in this specification and the appended claims, the singular forms “a”, “an” and “the” include plural referents unless the context clearly dictates otherwise. Thus, for example, reference to “a biopolymer” can include more than one biopolymer.

Definitions

The following definitions are provided for specific terms that are used in the following written description.

A “biopolymer” is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. As such, this term includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes deoxyribonucleic acid or DNA (including cDNA), ribonucleic acid or RNA and oligonucleotides, regardless of the source.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “mRNA” means messenger RNA.

A “biomonomer” references a single unit, which can be linked with the same or other biomonomers to form a biopolymer (for example, a single amino acid or nucleotide with two linking groups one or both of which may have removable protecting groups). A biomonomer fluid or biopolymer fluid reference a liquid containing either a biomonomer or biopolymer, respectively (typically in solution).

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Nucleotide sub-units of deoxyribonucleic acids are deoxyribonucleotides, and nucleotide sub-units of ribonucleic acids are ribonucleotides.

An “oligonucleotide” generally refers to a nucleotide multimer of about 10 to 100 nucleotides in length, while a “polynucleotide” or “nucleic acid” includes a nucleotide multimer having any number of nucleotides.

The term “base composition properties” shall refer to properties of a sequence related to base composition. By way of example, while not limiting the term, base composition properties can include the percentage of A, C, T, and G sequences within a given probe sequence.

The term “primary structural features” as used herein shall refer to structural features of a sequence related the contiguous positioning of bases in the sequence. While not limiting the term, an example of a primary structural feature is a homopolymeric run.

The term “homopolymeric run” as used herein shall refer to a portion of a base sequence wherein a given base is repeated more than once. By way of example, a sequence contains the contiguous bases “TTTTT” would be considered to have a homopolymeric run.

The term “secondary structural features” as used herein shall refer to structural features (predicted or empirical) of a sequence caused by the interaction between both contiguous and non-contiguous bases in the sequence. While not limiting the term, an example of a secondary structural feature is a hairpin loop structure.

As used herein, the term “thermodynamic characteristics” shall refer to characteristics of a sequence described in thermodynamic terms. By way of example, while not limiting the term, thermodynamic characteristics of a given sequence can include the Gibbs free energy of hybridization of that sequence with another sequence. As a further example, while not limiting the term, thermodynamic characteristics of a given sequence can include the melting temperature (Tm) of the sequence.

A chemical “array”, unless a contrary intention appears, includes any one, two or three-dimensional arrangement of addressable regions bearing a particular chemical moiety or moieties (for example, biopolymers such as polynucleotide sequences) associated with that region, where the chemical moiety or moieties are immobilized on the surface in that region. By “immobilized” is meant that the moiety or moieties are stably associated with the substrate surface in the region, such that they do not separate from the region under conditions of using the array, e.g., hybridization and washing and stripping conditions. As is known in the art, the moiety or moieties may be covalently or non-covalently bound to the surface in the region. For example, each region may extend into a third dimension in the case where the substrate is porous while not having any substantial third dimension measurement (thickness) in the case where the substrate is non-porous. An array may contain more than ten, more than one hundred, more than one thousand more than ten thousand features, or even more than one hundred thousand features, in an area of less than 20 cm² or even less than 10 cm². For example, features may have widths (that is, diameter, for a round spot) in the range of from about 10 μm to about 1.0 cm. In other embodiments each feature may have a width in the range of about 1.0 μm to about 1.0 mm, such as from about 5.0 μm to about 500 μm, and including from about 10 μm to about 200 μm. Non-round features may have area ranges equivalent to that of circular features with the foregoing width (diameter) ranges. A given feature is made up of chemical moieties, e.g., nucleic acids, that bind to (e.g., hybridize to) the same target (e.g., target nucleic acid), such that a given feature corresponds to a particular target. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide. Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature may incidentally detect non-targets of that feature). The target for which each feature is specific is, in representative embodiments, known. An array feature is generally homogenous in composition and concentration and the features may be separated by intervening spaces (although arrays without such separation can be fabricated).

The phrase “oligonucleotide bound to a surface of a solid support” or “probe bound to a solid support” or a “target bound to a solid support” refers to an oligonucleotide or mimetic thereof, e.g., PNA, LNA or UNA molecule that is immobilized on a surface of a solid substrate, where the substrate can have a variety of configurations, e.g., a sheet, bead, particle, slide, wafer, web, fiber, tube, capillary, microfluidic channel or reservoir, or other structure. In certain embodiments, the collections of oligonucleotide elements employed herein are present on a surface of the same planar support, e.g., in the form of an array. It should be understood that the terms “probe” and “target” are relative terms and that a molecule considered as a probe in certain assays may function as a target in other assays.

“Addressable sets of probes” and analogous terms refer to the multiple known regions of different moieties of known characteristics (e.g., base sequence composition) supported by or intended to be supported by an array surface, such that each location is associated with a moiety of a known characteristic and such that properties of a target moiety can be determined based on the location on the array surface to which the target moiety binds under stringent conditions.

In certain embodiments, an array is contacted with a nucleic acid sample under stringent assay conditions, i.e., conditions that are compatible with producing bound pairs of biopolymers of sufficient affinity to provide for the desired level of specificity in the assay while being less compatible to the formation of binding pairs between binding members of insufficient affinity. Stringent assay conditions are the summation or combination (totality) of both binding conditions and wash conditions for removing unbound molecules from the array.

As known in the art, “stringent hybridization conditions” and “stringent hybridization wash conditions” in the context of nucleic acid hybridization are sequence dependent, and are different under different experimental parameters. Stringent hybridization conditions include, but are not limited to, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization in 0.5 M NaHPO4, 7% sodium dodecyl sulfate (SDS), 1 mM EDTA at 65° C., and washing in 0.1×SSC/0.1% SDS at 68° C. can be performed. Additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

Wash conditions used to remove unbound nucleic acids may include, e.g., a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature. Other methods of agitation can be used, e.g., shaking, spinning, and the like.

Stringent hybridization conditions may also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with Cot-1 DNA, or the like.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate. The term “highly stringent hybridization conditions” as used herein refers to conditions that are compatible to produce complexes between complementary binding members, i.e., between immobilized probes and complementary sample nucleic acids, but which does not result in any substantial complex formation between non-complementary nucleic acids (e.g., any complex formation which cannot be detected by normalizing against background signals to interfeature areas and/or control regions on the array).

Additional hybridization methods are described in references describing CGH techniques (Kallioniemi et al., Science 1992; 258:818-821 and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, Gall et al. Meth. Enzymol. 1981; 21:470-480 and Angerer et al., In Genetic Engineering: Principles and Methods, Setlow and Hollaender, Eds. Vol 7, pgs 43-65 (Plenum Press, New York 1985). See also U.S. Pat. Nos: 6,335,167; 6,197,501; 5,830,645; and 5,665,549; the disclosures of which are herein incorporated by reference.

The term “sample” as used herein relates to a material or mixture of materials, containing one or more components of interest. Samples include, but are not limited to, samples obtained from an organism or from the environment (e.g., a soil sample, water sample, etc.) and may be directly obtained from a source (e.g., such as a biopsy or from a tumor) or indirectly obtained e.g., after culturing and/or one or more processing steps. In one embodiment, samples are a complex mixture of molecules, e.g., comprising at least about 50 different molecules, at least about 100 different molecules, at least about 200 different molecules, at least about 500 different molecules, at least about 1000 different molecules, at least about 5000 different molecules, at least about 10,000 molecules, etc.

As used herein, a “biologically occurring sequence” refers to a sequence in a biological sample of target nucleic acids, e.g., such as a sequence from a biological organism, cell, tissue type, etc., being evaluated by hybridization to a collection of probe molecules which are designed to detect one or more sequences in the biological sample (e.g., by specifically hybridizing to the sequence under stringent conditions). Probes with no significant similarity to a biologically occurring sequence are those which are selected (e.g., by methods as described herein) not to hybridize to the sequences under stringent conditions such that they can be used as negative controls for test probes which are designed to detect the one or more sequences in the biological sample.

The term “genome” refers to all nucleic acid sequences (coding and non-coding) and elements present in any virus, single cell (prokaryote and eukaryote) or each cell type in a metazoan organism. The term genome also applies to any naturally occurring or induced variation of these sequences that may be present in a mutant or disease variant of any virus or cell or cell type. Genomic sequences include, but are not limited to, those involved in the maintenance, replication, segregation, and generation of higher order structures (e.g. folding and compaction of DNA in chromatin and chromosomes), or other functions, if any, of nucleic acids, as well as all the coding regions and their corresponding regulatory elements needed to produce and maintain each virus, cell or cell type in a given organism.

For example, the human genome consists of approximately 3.0×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements and amplification of any subchromosomal region or DNA sequence. In certain aspects, a “genome” refers to nuclear nucleic acids, excluding mitochondrial nucleic acids; however, in other aspects, the term does not exclude mitochondrial nucleic acids. In still other aspects, the “mitochondrial genome” is used to refer specifically to nucleic acids found in mitochondrial fractions.

An “array layout” or “array characteristics”, refers to one or more physical, chemical or biological characteristics of the array, such as positioning of some or all the features within the array and on a substrate, one or more feature dimensions, or some indication of an identity or function (for example, chemical or biological) of a moiety at a given location, or how the array should be handled (for example, conditions under which the array is exposed to a sample, or array reading specifications or controls following sample exposure).

As used herein, a “test nucleic acid sample” or “test nucleic acids” refer to nucleic acids comprising sequences whose quantity or degree of representation (e.g., copy number) or sequence identity is being assayed. Similarly, “test genomic acids” or a “test genomic sample” refers to genomic nucleic acids comprising sequences whose quantity or degree of representation (e.g., copy number) or sequence identity is being assayed.

As used herein, a “reference nucleic acid sample” or “reference nucleic acids” refers to nucleic acids comprising sequences whose quantity or degree of representation (e.g., copy number) or sequence identity is known. Similarly, “reference genomic acids” or a “reference genomic sample” refers to genomic nucleic acids comprising sequences whose quantity or degree of representation (e.g., copy number) or sequence identity is known. A “reference nucleic acid sample” may be derived independently from a “test nucleic acid sample,” i.e., the samples can be obtained from different organisms or different cell populations of the sample organism. However, in certain embodiments, a reference nucleic acid is present in a “test nucleic acid sample” which comprises one or more sequences whose quantity or identity or degree of representation in the sample is unknown while containing one or more sequences (the reference sequences) whose quantity or identity or degree of representation in the sample is known. The reference nucleic acid may be naturally present in a sample (e.g., present in the cell from which the sample was obtained) or may be added to or spiked in the sample.

If a surface-bound polynucleotide or probe “corresponds to” a chromosome, the polynucleotide usually contains a sequence of nucleic acids that is unique to that chromosome. Accordingly, a surface-bound polynucleotide that corresponds to a particular chromosome usually specifically hybridizes to a labeled nucleic acid made from that chromosome, relative to labeled nucleic acids made from other chromosomes. Array features, because they usually contain surface-bound polynucleotides, can also correspond to a chromosome.

A “non-cellular chromosome composition” is a composition of chromosomes synthesized by mixing pre-determined amounts of individual chromosomes. These synthetic compositions can include selected concentrations and ratios of chromosomes that do not naturally occur in a cell, including any cell grown in tissue culture. Non-cellular chromosome compositions may contain more than an entire complement of chromosomes from a cell, and, as such, may include extra copies of one or more chromosomes from that cell. Non-cellular chromosome compositions may also contain less than the entire complement of chromosomes from a cell.

A “CGH array” or “aCGH array” refers to an array that can be used to compare DNA samples for relative differences in copy number. In general, an aCGH array can be used in any assay in which it is desirable to scan a genome with a sample of nucleic acids. For example, an aCGH array can be used in location analysis as described in U.S. Pat. No. 6,410,243, the entirety of which is incorporated herein and thus can also be referred to as a “location analysis array” or an “array for ChIP-chip analysis.” In certain aspects, a CGH array provides probes for screening or scanning a genome of an organism and comprises probes from a plurality of regions of the genome. In one aspect, the array comprises probe sequences for scanning an entire chromosome arm, wherein probes targets are separated by at least about 500 bp, at least about 1 kb, at least about 5 kb, at least about 10 kb, at least about 25 kb, at least about 50 kb, at least about 100 kb, at least about 250 kb, at least about 500 kb and at least about 1 Mb. In another aspect, the array comprises probes sequences for scanning an entire chromosome, a set of chromosomes, or the complete complement of chromosomes forming the organism's genome. By “resolution” is meant the spacing on the genome between sequences found in the probes on the array. In some embodiments (e.g., using a large number of probes of high complexity) all sequences in the genome can be present in the array. The spacing between different locations of the genome that are represented in the probes may also vary, and may be uniform, such that the spacing is substantially the same between sampled regions, or non-uniform, as desired. An assay performed at low resolution on one array, e.g., comprising probe targets separated by larger distances, may be repeated at higher resolution on another array, e.g., comprising probe targets separated by smaller distances.

In certain aspects, in constructing the arrays, both coding and non-coding genomic regions are included as probes, whereby “coding region” refers to a region comprising one or more exons that is transcribed into an mRNA product and from there translated into a protein product, while by non-coding region is meant any sequences outside of the exon regions, where such regions may include regulatory sequences, e.g., promoters, enhancers, untranslated but transcribed regions, introns, origins of replication, telomeres, etc. In certain embodiments, one can have at least some of the probes directed to non-coding regions and others directed to coding regions. In certain embodiments, one can have all of the probes directed to non-coding sequences and such sequences can, optionally, be all non-transcribed sequences (e.g., intergenic regions including regulatory sequences such as promoters and/or enhancers lying outside of transcribed regions).

In certain aspects, an array may be optimized for one type of genome scanning application compared to another, for example, the array can be enriched for intergenic regions compared to coding regions for a location analysis application.

In some embodiments, at least 5% of the polynucleotide probes on the solid support hybridize to regulatory regions of a nucleotide sample of interest while other embodiments may have at least 30% of the polynucleotide probes on the solid support hybridize to exonic regions of a nucleotide sample of interest. In yet other embodiments, at least 50% of the polynucleotide probes on the solid support hybridize to intergenic regions (e.g., non-coding regions which exclude introns and untranslated regions, i.e., comprise non-transcribed sequences) of a nucleotide sample of interest.

In certain aspects, probes on the array represent random selection of genomic sequences (e.g., both coding and noncoding). However, in other aspects, particular regions of the genome are selected for representation on the array, e.g., such as CpG islands, genes belonging to particular pathways of interest or whose expression and/or copy number are associated with particular physiological responses of interest (e.g., disease, such a cancer, drug resistance, toxological responses and the like). In certain aspects, where particular genes are identified as being of interest, intergenic regions proximal to those genes are included on the array along with, optionally, all or portions of the coding sequence corresponding to the genes. In one aspect, at least about 100 bp, 500 bp, 1,000 bp, 5,000 bp, 10,000 kb or even 100,000 kb of genomic DNA upstream of a transcriptional start site is represented on the array in discrete or overlapping sequence probes. In certain aspects, at least one probe sequence comprises a motif sequence to which a protein of interest (e.g., such as a transcription factor) is known or suspected to bind.

In certain aspects, repetitive sequences are excluded as probes on the arrays. However, in another aspect, repetitive sequences are included.

The choice of nucleic acids to use as probes may be influenced by prior knowledge of the association of a particular chromosome or chromosomal region with certain disease conditions. International Application WO 93/18186 provides a list of exemplary chromosomal abnormalities and associated diseases, which are described in the scientific literature. Alternatively, whole genome screening to identify new regions subject to frequent changes in copy number can be performed using the methods of the present invention discussed further below.

In some embodiments, previously identified regions from a particular chromosomal region of interest are used as probes. In certain embodiments, the array can include probes which “tile” a particular region (e.g., which have been identified in a previous assay or from a genetic analysis of linkage), by which is meant that the probes correspond to a region of interest as well as genomic sequences found at defined intervals on either side, i.e., 5′ and 3′ of, the region of interest, where the intervals may or may not be uniform, and may be tailored with respect to the particular region of interest and the assay objective. In other words, the tiling density may be tailored based on the particular region of interest and the assay objective. Such “tiled” arrays and assays employing the same are useful in a number of applications, including applications where one identifies a region of interest at a first resolution, and then uses tiled array tailored to the initially identified region to further assay the region at a higher resolution, e.g., in an iterative protocol.

In certain aspects, the array includes probes to sequences associated with diseases associated with chromosomal imbalances for prenatal testing. For example, in one aspect, the array comprises probes complementary to all or a portion of chromosome 21 (e.g., Down's syndrome), all or a portion of the X chromosome (e.g., to detect an X chromosome deficiency as in Turner's Syndrome) and/or all or a portion of the Y chromosome Klinefelter Syndrome (to detect duplication of an X chromosome and the presence of a Y chromosome), all or a portion of chromosome 7 (e.g., to detect William's Syndrome), all or a portion of chromosome 8 (e.g., to detect Langer-Giedon Syndrome), all or a portion of chromosome 15 (e.g., to detect Prader-Willi or Angelman's Syndrome, all or a portion of chromosome 22 (e.g., to detect Di George's syndrome).

Other “themed” arrays may be fabricated, for example, arrays including whose duplications or deletions are associated with specific types of cancer (e.g., breast cancer, prostate cancer and the like). The selection of such arrays may be based on patient information such as familial inheritance of particular genetic abnormalities. In certain aspects, an array for scanning an entire genome is first contacted with a sample and then a higher-resolution array is selected based on the results of such scanning.

Themed arrays also can be fabricated for use in gene expression assays, for example, to detect expression of genes involved in selected pathways of interest, or genes associated with particular diseases of interest.

In one embodiment, a plurality of probes on the array are selected to have a duplex Tₘ within a predetermined range. For example, in one aspect, at least about 50% of the probes have a duplex Tₘ within a temperature range of about 75° C. to about 85° C. In one embodiment, at least 80% of said polynucleotide probes have a duplex Tₘ within a temperature range of about 75° C. to about 85° C., within a range of about 77° C. to about 83° C., within a range of from about 78° C. to about 82° C. or within a range from about 79° C. to about 82° C. In one aspect, at least about 50% of probes on an array have range of Tₘ's of less than about 4° C., less then about 3° C., or even less than about 2° C., e.g., less than about 1.5° C., less than about 1.0° C. or about 0.5° C.

The probes on the microarray, in certain embodiments have a nucleotide length in the range of at least 30 nucleotides to 200 nucleotides, or in the range of at least about 30 to about 150 nucleotides. In other embodiments, at least about 50% of the polynucleotide probes on the solid support have the same nucleotide length, and that length may be about 60 nucleotides.

In still other aspects, probes on the array comprise at least coding sequences.

In one aspect, probes represent sequences from an organism such as Drosophila melanogaster, Caenorhabditis elegans, yeast, zebrafish, a mouse, a rat, a domestic animal, a companion animal, a primate, a human, etc. In certain aspects, probes representing sequences from different organisms are provided on a single substrate, e.g., on a plurality of different arrays.

A “CGH assay” using an aCGH array can be generally performed as follows. In one embodiment, a population of nucleic acids contacted with an aCGH array comprises at least two sets of nucleic acid populations, which can be derived from different sample sources. For example, in one aspect, a target population contacted with the array comprises a set of target molecules from a reference sample and from a test sample. In one aspect, the reference sample is from an organism having a known genotype and/or phenotype, while the test sample has an unknown genotype and/or phenotype or a genotype and/or phenotype that is known and is different from that of the reference sample. For example, in one aspect, the reference sample is from a healthy patient while the test sample is from a patient suspected of having cancer or known to have cancer.

In one embodiment, a target population being contacted to an array in a given assay comprises at least two sets of target populations that are differentially labeled (e.g., by spectrally distinguishable labels). In one aspect, control target molecules in a target population are also provided as two sets, e.g., a first set labeled with a first label and a second set labeled with a second label corresponding to first and second labels being used to label reference and test target molecules, respectively.

In one aspect, the control target molecules in a population are present at a level comparable to a haploid amount of a gene represented in the target population. In another aspect, the control target molecules are present at a level comparable to a diploid amount of a gene. In still another aspect, the control target molecules are present at a level that is different from a haploid or diploid amount of a gene represented in the target population. The relative proportions of complexes formed labeled with the first label vs. the second label can be used to evaluate relative copy numbers of targets found in the two samples.

In certain aspects, test and reference populations of nucleic acids may be applied separately to separate but identical arrays (e.g., having identical probe molecules) and the signals from each array can be compared to determine relative copy numbers of the nucleic acids in the test and reference populations.

Methods to fabricate arrays are described in detail in U.S. Pat. Nos. 6,242,266; 6,232,072; 6,180,351; 6,171,797 and 6,323,043. As already mentioned, these references are incorporated herein by reference. Drop deposition methods can be used for fabrication, as previously described herein. Also, instead of drop deposition methods, photolithographic array fabrication methods may be used. Interfeature areas need not be present particularly when the arrays are made by photolithographic methods as described in those patents.

Following receipt by a user, an array will typically be exposed to a sample and then read. Reading of an array may be accomplished by illuminating the array and reading the location and intensity of resulting fluorescence at multiple regions on each feature of the array. For example, a scanner may be used for this purpose is the AGILENT MICROARRAY SCANNER manufactured by Agilent Technologies, Palo, Alto, Calif. or other similar scanner. Other suitable apparatus and methods are described in U.S. Pat. Nos. 6,518,556; 6,486,457; 6,406,849; 6,371,370; 6,355,921; 6,320,196; 6,251,685 and 6,222,664. Scanning typically produces a scanned image of the array which may be directly inputted to a feature extraction system for direct processing and/or saved in a computer storage device for subsequent processing. However, arrays may be read by any other methods or apparatus than the foregoing, other reading methods including other optical techniques or electrical techniques (where each feature is provided with an electrode to detect bonding at that feature in a manner disclosed in U.S. Pat. Nos. 6,251,685, 6,221,583 and elsewhere).

It should also be noted that, as used in this specification and the appended claims, the term “configured” describes a system, apparatus, or other structure that is constructed or configured to perform a particular task or adopt a particular configuration to. The phrase “configured” can be used interchangeably with other similar phrases such as arranged and configured, constructed and arranged, adapted, constructed, manufactured and arranged, and the like.

Various embodiments disclosed herein can be used to generate negative control probe sequences. The term “negative control probe sequence” as used herein includes sequences of bases that can be deposited on an array and serve as a negative control during use of the array. The sequence is not limited by the type of application being performed, i.e., the sequence can be designed for arrays designed for any of a variety of uses, e.g., gene expression analysis, mutation analysis, sequencing, genotyping, comparative genome hybridization analysis, location analysis (e.g., ChIP-chip analysis), and genome scanning applications generally.

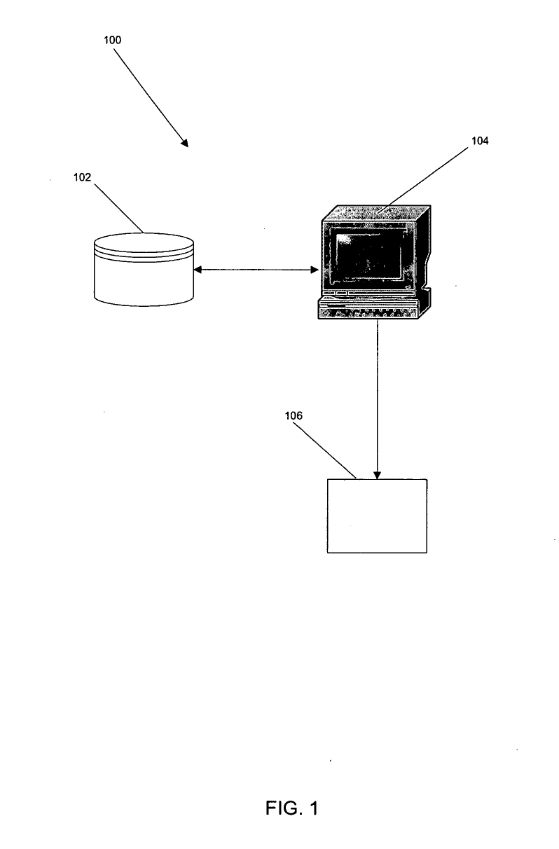

Referring now to FIG. 1, a schematic diagram of an exemplary system 100 for manufacturing arrays is shown. A computing system 104 is in electronic communication with a database 102 and an array printer 106. In an embodiment, the computing system 104 directs the operations of the array printer 106. It will be appreciated that in some embodiments the computing system 104 is part of the array printer 106. However, in other exemplary embodiments, the computing system 104 and the array printer 106 are separate. In addition, it will be appreciated that in some embodiments the database 102 is part of the computing system 104. However, in other exemplary embodiments, the database 102 and the computing system 104 are separate. The computing system 104 can query the database 102 as desired to retrieve data on probe sequences or on known sequences.

The array printer 106 can perform various steps to generate features of biopolymer probes (e.g., nucleic acids) on the array substrate. Exemplary array manufacturing machines and methods are described in U.S. Pat. Nos. 6,900,048; 6,890,760; 6,884,580; and 6,372,483. In some embodiments, the array printer 106 uses inkjet technology. In an embodiment, the array printer 106 prints spots of pre-synthesized nucleotide sequences onto the array substrate. In an embodiment, the array printer 106 can be used for in situ fabrication, where nucleotide sequences are built on the array one base at a time. Embodiments of the array printer 106 can also include those that use photolithographic methods to deposit nucleotide sequences onto the array substrates. Some embodiments of methods described herein are performed as a part of the array manufacturing process. However, other embodiments of methods described herein are performed separately from the array manufacturing process.

Some embodiments described herein are implemented as logical operations in a computing system, such as the computing system 104. The logical operations can be implemented (1) as a sequence of computer implemented steps or program modules running on a computer system and (2) as interconnected logic or hardware modules running within the computing system. This implementation is a matter of choice dependent on the performance requirements of the specific computing system. Accordingly, the logical operations making up the embodiments described herein are referred to as operations, steps, or modules. It will be recognized by one of ordinary skill in the art that these operations, steps, and modules may be implemented in software, in firmware, in special purpose digital logic, and any combination thereof without deviating from the spirit and scope of the claims attached hereto. This software, firmware, or similar sequence of computer instructions may be encoded and stored upon computer readable storage medium and may also be encoded within a carrier-wave signal for transmission between computing devices.

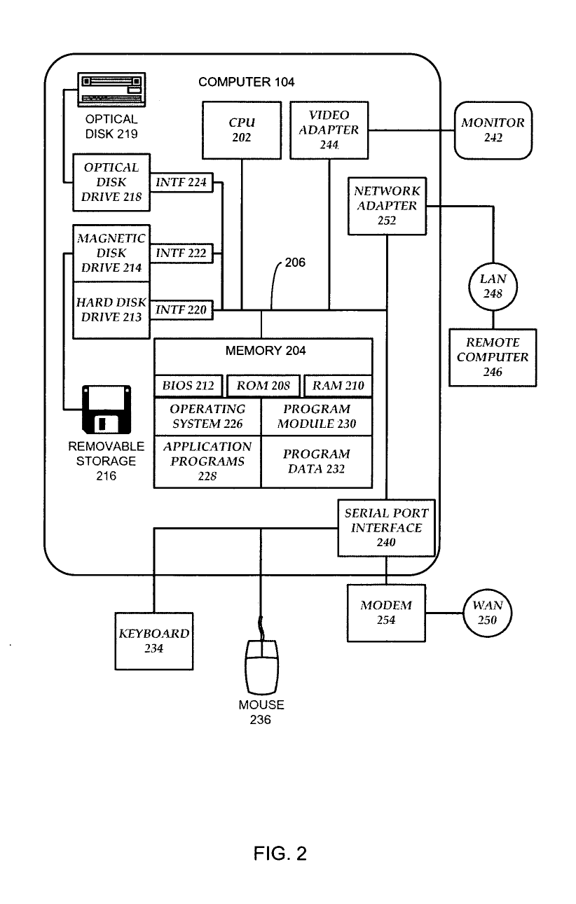

Referring now to FIG. 2, an example computing system 104 is illustrated. The computing system 104 illustrated in FIG. 2 can take a variety of forms such as, for example, a mainframe, a desktop computer, a laptop computer, a hand-held computer, or any other programmable device. In addition, although computing system 104 is illustrated, the systems and methods disclosed herein can be implemented in various alternative computer systems as well.

The computing system 104 includes a processor unit 202, a system memory 204, and a system bus 206 that couples various system components including the system memory 204 to the processor unit 202. The system bus 206 can be any of several types of bus structures including a memory bus, a peripheral bus and a local bus using any of a variety of bus architectures. The system memory includes read only memory (ROM) 208 and random access memory (RAM) 210. A basic input/output system 212 (BIOS), which contains basic routines that help transfer information between elements within the computing system 104, is stored in ROM 208.

The computing system 104 further includes a hard disk drive 213 for reading from and writing to a hard disk, a magnetic disk drive 214 for reading from or writing to a removable magnetic disk 216, and an optical disk drive 218 for reading from or writing to a removable optical disk 219 such as a CD ROM, DVD, or other optical media. The hard disk drive 213, magnetic disk drive 214, and optical disk drive 218 are connected to the system bus 206 by a hard disk drive interface 220, a magnetic disk drive interface 222, and an optical drive interface 224, respectively. The drives and their associated computer-readable media provide nonvolatile storage of computer readable instructions, data structures, programs, and other data for the computing system 104.

Although the example environment described herein can employ a hard disk 213, a removable magnetic disk 216, and a removable optical disk 219, other types of computer-readable media capable of storing data can be used in the example system 104. Examples of these other types of computer-readable mediums that can be used in the example operating environment include magnetic cassettes, flash memory cards, digital video disks, Bernoulli cartridges, random access memories (RAMs), and read only memories (ROMs).

A number of program modules can be stored on the hard disk 213, magnetic disk 216, optical disk 219, ROM 208, or RAM 210, including an operating system 226, one or more application programs 228, other program modules 230, and program data 232.

A user may enter commands and information into the computing system 104 through input devices such as, for example, a keyboard 234, mouse 236, or other pointing device. These and other input devices are often connected to the processing unit 202 through a serial port interface 240 that is coupled to the system bus 206. Nevertheless, these input devices also may be connected by other interfaces, such as a parallel port, game port, or a universal serial bus (USB). An LCD display 242 or other type of display device is also connected to the system bus 206 via an interface, such as a video adapter 244.

The computer system 104 may operate in a networked environment using logical connections to one or more remote computers, such as a remote computer 246. The remote computer 246 may be a computer system, a server, a router, a network PC, a peer device or other common network node, and typically includes many or all of the elements described above relative to the computer system 104. The network connections include a local area network (LAN) 248 and a wide area network (WAN) 250. When used in a LAN networking environment, the computer system 104 is connected to the local network 248 through a network interface or adapter 252. When used in a WAN networking environment, the computing system 104 typically includes a modem 254 or other means for establishing communications over the wide area network 250, such as the Internet. In a networked environment, program modules depicted relative to the computing system 104, or portions thereof, may be stored in the remote memory storage device. It will be appreciated that the network connections shown are examples and other means of establishing a communications link between the computers may be used.

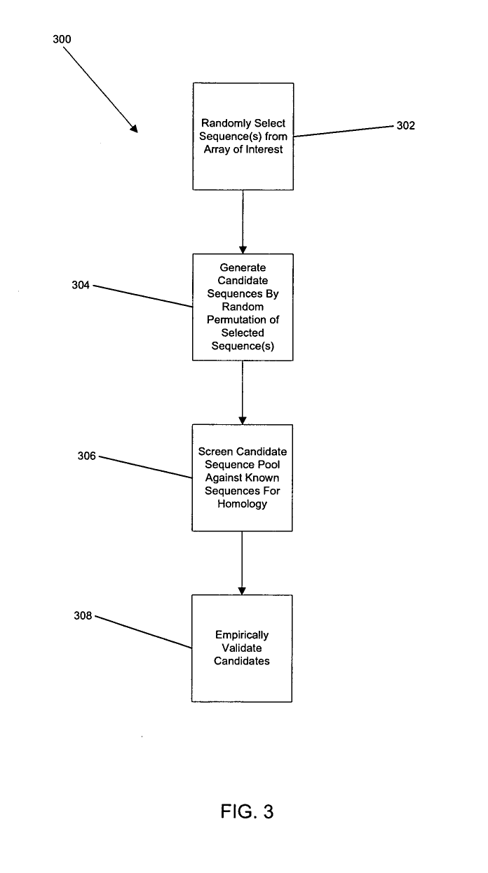

Referring now to FIG. 3, a flowchart 300 is provided illustrating operations that are performed in some embodiments. First, one or more biological probe sequences of interest are randomly selected from an array of interest 302. As used herein, the term “biological probe sequences” includes those sequences of a set of sequences that are designed to hybridize with target molecules (also referred to as biologically occurring molecules), such as nucleotide sequences, that may be present in a sample. Such sequences may be included on a chemical array. Next, a pool of candidate sequences is generated by randomly permuting the bases (or nucleotides) of each selected biological probe sequences 304. The term “permuting” as used herein shall mean to change the order or arrangement of bases within a sequence. One or more screening operations are then performed on the pool of candidate sequences. As an example of one screening operation, the candidate sequences are screened for similarity against known biological sequences of genome or transcriptome of the organism of interest to eliminate those having significant similarity with any known biological sequence 306. The best-alignment of a 60-mer negative control sequence to the human genomic sequence should contain no contiguous hits of more than 20 consecutive bases or about 33% of the probe sequence as determined by a BLAST search using default parameters. Using ProbeSpec with a index-seed size of 10 there should be hits with fewer than 20 mismatches across the length of the probe for the nearest hit in the genome.

The organism of interest is the organism, or any of the organisms, for which the array is designed to analyze samples from. Individual screening operations are performed by themselves or in addition to other screening operations. Then, in some embodiments, the remaining candidate sequences are empirically validated on a test array 308. For example, candidate sequences can be synthesized and then put on a test array (or synthesized in situ) and then the candidate sequences can be tested for hybridization with a test sample. Operations performed in some embodiments will now be discussed in greater detail.

Some embodiments include random selection of biological probe sequences from a set of sequences (e.g., such as a plurality of sequences designed for inclusion on a chemical array of interest). The array of interest is the particular array for which negative control probes are being designed. The selected biological probe sequences then serve as the starting point from which candidate probe sequences are generated (as further described below). In one aspect, when biological probe sequences are used as the starting point, the resulting candidate probe sequences will match the base composition (e.g., A/T/G/C %) of the biological probe sequences in the array of interest. In certain aspects, the resulting candidate probes can be used to more accurately measure both residual spatially varying background as well as the sequence specific background variations. In certain aspects, by randomly choosing the biological probes to use for generating the candidate probes, the resulting negative control probe sequences have base compositions and thermodynamic properties that closely represent those distributions for the biological probes themselves.

In certain aspects, screening can include screening the candidate sequences for base composition properties such as for A/C/T/G content, the presence or absence of homopolymeric runs, screening for hairpin loops or for thermodynamic characteristics such as for melting temperature. In general, each screening operation reduces the pool of potential candidate sequences. Methods of screening according to such characteristics are described in U.S. patent application Ser. No. 11/232,817, filed Sep. 21, 2005, incorporated by reference herein.

Arrays can include any desired number of biological probe sequences. By way of example, arrays can include 10s, 100s, 1,000s, or 10,000s of different biological probe sequences. Any desired number of the biological probe sequences can be randomly selected. The desired number may depend on the number of biological probe sequences in the array of interest. In some embodiments, the number of biological probe sequences selected is equal to between about 0.1% and 20% of the biological probe sequences on the array of interest.

It will be appreciated there are many ways of randomly selecting individuals from among a group. By way of example, different biological probe sequences can be assigned different reference numbers and then a subset of the reference numbers can be randomly or pseudo-randomly selected. The term “random” as used herein shall include pseudo-random unless indicated to the contrary. Techniques of random number selection can include lottery methods, the use of random number tables, entropy approaches, and the like. It will also be appreciated that there are many ways of using computer systems to automatically generate random numbers. Further, techniques for generating random numbers can be implemented in many different programming languages. After random selection of biological probe sequences, the selected sequences are then used as the starting point for candidate probe generation.

In some embodiments, nucleotide base sequences are represented by the letters A/T/G/C. It will be appreciated that these letters correspond to the bases occurring in DNA (adenine, thymine, guanine, and cytosine). However, in some embodiments, other letters are used corresponding to components of other biopolymers, such as RNA or polypeptides. In addition, in some embodiments, letters are used corresponding to artificial components such as non-naturally occurring bases or peptides. As used herein the term “bases” or “monomer units” or “letters” may be used interchangeably though in specific contexts as will be apparent, the term “bases” or “monomer units” will refer to the chemical moieties, while “letters” will refer to a representation of the former.

Some embodiments include generating candidate probe sequences. The term “candidate probe sequences” as used herein includes generated sequences that are later subject to one or more screening steps in order to produce negative control probe sequences. Biological probe sequences selected from an array of interest can serve as the starting point for the generation of a pool of candidate probe sequences. By way of example, the selected biological probe sequences can be randomly permuted to form a pool of candidate probe sequences. There are many techniques of random sequence permutation that can be used. By way of example, the letters (corresponding to bases) of a given selected biological probe sequence can be tallied with regard to the total number of each letter present. By way of example, assuming the selected biological probe sequences are 60 bases in length, a given selected biological probe sequence may be found to contain the following composition of bases: 13 A, 16 T, 15 G, and 16 C. A permuted random sequence can then be generated using this group of letters by randomly selecting one letter out of the group for each position in the permuted sequence until all of the 60 letters are used. In this case, the resulting permuted sequence would still contain a total 60 letters (specifically 13 A, 16 T, 15 G, and 16 C) but the sequence of letters would be different than the sequence of letters in the original selected biological probe sequence. It will be appreciated that there are many other techniques that can be used for generating random permuted sequences based on a given starting sequence.

The total number of possible unique random permutations depends on the total length of the sequence and the composition of different letters within the sequence. However, in the example of a sequence that is 60 bases in length having a relatively even distribution of bases, it will be appreciated that a very large number of random permutations are possible. It is estimated that only a fraction of these randomly generated permutation sequences are found within the sequences of all living organisms. An even smaller fraction would be found with the sequences of a given organism, such as the organism of interest. For any given length of random sequence generated, those that are found within the sequences of the organism of interest can be removed from the candidate pool through similarity screening, in silico, as described further below and/or by empirical testing (e.g., in a hybridization experiment).

In some embodiments, the pool of candidate sequences generated is screened for sequence similarity against the entire genome (for CGH arrays or arrays used for location analysis, e.g., ChIP-chip analysis) or the entire transcriptome (for expression arrays) of an organism from which samples to be tested will be obtained (organism of interest). The term “sequence similarity” as used herein shall refer to the degree to which two sequences are similar in their base sequence. Sequence similarity can be quantified in various ways known to those of skill in the art. Eliminating candidate sequences from the pool that have substantial similarity to sequences of an organism of interest helps to ensure that candidate sequences will be chosen that will function as negative controls. Similarity screening can be performed using many different tools available to those of skill in the art. A possible example includes determining similarity using the BLASTN program available at the website for the National Center for Biotechnology Information (NCBI). The BLASTN program uses the heuristic search algorithm BLAST (Basic Local Alignment Search Tool) to compare a nucleotide sequence (N) against a nucleotide sequence dataset. See Altschul et al., 1990, J. Mol. Biol., 215:403-10. The BLAST algorithm identifies regions of local similarity and then moves bi-directionally until the BLAST score declines. Another useful tool is BLAT. See Kent W J. BLAT-The BLAST-Like Alignment Tool. Genome Research, April 12(4):656-64. 2002. ProbeSpec is another useful tool that calculates the numbers of mismatches of nearest hits. See Doron Lipson, Peter Web, Zohar Yakhini (2002) “Designing Specific Oligonucleotide Probes for the Entire S. cerevisiae Transcriptome”, WABI '02, 17-21/9/02, Rome.

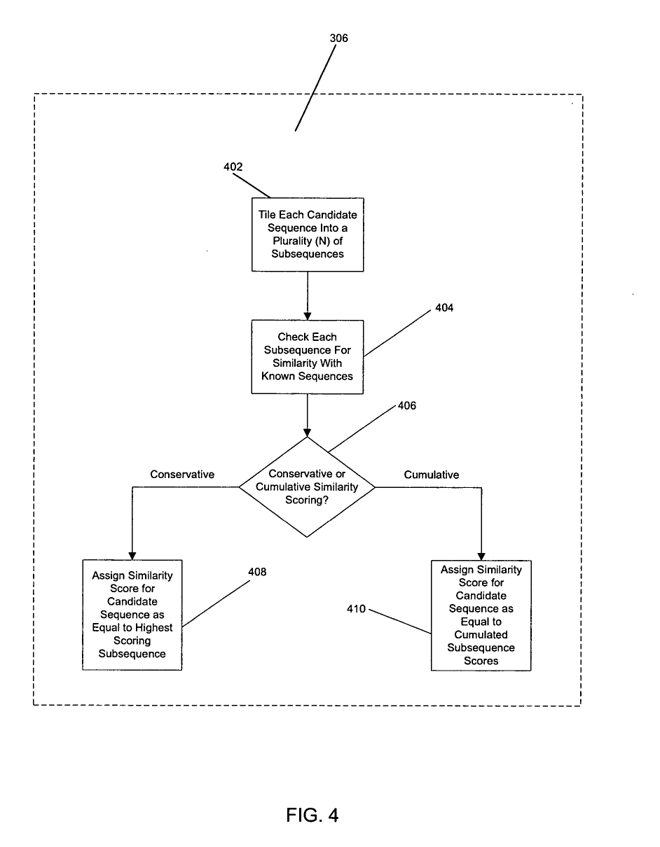

In some embodiments, subsequences of candidate sequences are screened for similarity against known biological sequences of an organism (or organisms) of interest. Referring now to FIG. 4, in an embodiment, a given candidate sequence can be subdivided into a plurality of overlapping or non-overlapping subsequences 402, each of which is then screened for similarity against known biological sequences of an organism of interest 404. For example, a candidate sequence having a length of 60 bases could be subdivided into three distinct subsequences wherein the first subsequence comprises bases 1-30 of the candidate sequence, the second subsequence comprises bases 15-45 of the candidate sequence, and the third subsequence comprises bases 30-60 of the candidate sequence. Then each of these subsequences can be compared with a database of known sequences to check for significant similarity 404. It is believed that screening subsequences can offer advantages in that it can make it less likely that any sub-region within a given candidate sequence has a significant match from within the genome or transcriptome of the organism of interest. However, in some embodiments similarity screening is performed using the full candidate sequences.

Similarity can be scored in various ways. In one embodiment, histograms showing the closest matches found are prepared for each sequence or subsequences. Specifically, a histogram is generated showing the number of hits as a function of “distance” of candidate sequences or subsequences from known sequences within the genome or transcriptome of the organism of interest. For example, a distance of 0 base pair(s) corresponds to a candidate sequence that has a direct match in the known sequences within the genome or transcriptome of the organism of interest. Similarly, a distance of 1 base pair(s) corresponds to a candidate sequence having a match in the known sequences within the genome or transcriptome of the organism of interest that is different by only 1 base. Then a score is assigned based on the histogram with “smaller distance” hits (more similar) increasing the score more than “longer distance” hits (less similar). For example, each hit with a distance of 1 base pair might result in increasing the total score for the candidate sequence by 15 units whereas each hit with a distance of 2 base pairs might result in increasing the total score for the candidate sequence by only 12 units. This is only one example of how similarity can be scored. It will be appreciated that scoring can be conducted in many different ways as desired.

In the example of similarity screening performed on subsequences after subdividing the candidate sequences, scoring can be tallied in either a conservative or cumulative manner (see decision 406 in FIG. 4). In an embodiment of the conservative approach 408, scoring can be done by calculating the distribution of similarity scores for each of the subdivided subsequences from a given candidate sequence. Then, the subsequence having the highest similarity score to any sequence from the organism of interest is used to set the score for the overall candidate sequence from which the subsequences are taken. For example, if there are three subsequences in a given candidate sequence and one of the sequences has a score that is higher than the other two, then that higher score is taken as the score for the whole candidate sequence.

Alternatively, similarity scoring for candidate sequences can be done in a cumulative manner. In an embodiment of the cumulative approach 410, the similarity scores for each subsequence are calculated and then cumulated or averaged. For example, assuming there are 3 subsequences for a given candidate sequence and each subsequence produces similarity scores of X, Y, and Z respectively, then the similarity score for the given candidate sequence can be set as either the sum of X, Y, and Z or the average of X, Y, and Z. While some specific examples of calculating similarity scores for candidate sequences have been illustrated herein, it will be appreciated that there are many other ways of calculating similarity scores.

After similarity scores are calculated for candidate sequences, those sequences resulting in scores that indicate significant similarity with one or more naturally occurring sequences in the genome or transcriptome of the organism of interest are removed from the candidate sequence pool. The precise cut-off level for similarity scores will depend on various factors including the length of the candidate sequences, the stringency of wash steps used in the hybridization protocol for the array of interest, scoring method, etc.

Candidate probe sequences that have significant similarity to naturally occurring sequences are undesirable for use as negative controls. In some embodiments, a BLAST raw score (S) is used to select those sequences that do not have significant similarity to known biological sequences. It will be appreciated that BLAST raw score thresholds can be set as desired. In an embodiment, candidate negative control sequences producing any matches against biological sequences with a BLAST raw score of greater than or equal to about 20 are not used. In an embodiment, candidate negative control sequences producing any matches against biological sequences with a BLAST raw score of greater than or equal to about 25 are not used. In an embodiment, candidate negative control sequences producing any matches against biological sequences with a BLAST raw score of greater than or equal to about 30 are not used. In an embodiment, candidate negative control sequences producing any matches against biological sequences with a BLAST raw score of greater than or equal to about 30.23 are not used.

In an embodiment, candidate sequences predicted to form a hybrid with any naturally occurring sequence in the genome or transcriptome of the organism of interest having a predicted Tₘ sufficiently high that the hybrid would be predicted not to melt off during the most stringent post-hybridization was step used in the hybridization protocol are removed from the candidate sequence pool. In some embodiments, candidate sequences having sequence identity of greater than 10 contiguous complementary base pairs, or equally stable longer homologous sequences containing deletions or mismatches, are removed from the candidate sequence pool. In some embodiments, candidate sequences having sequence identity of greater than 15 contiguous complementary base pairs, or equally stable longer homologous sequences containing deletions or mismatches, are removed from the candidate sequence pool.

Closely related to similarity screening, some embodiments can include screening candidate probes for hybridization potential. Hybridization potentials can be calculated using various algorithms known to those of skill in the art. By way of example, hybridization potentials for given sequences can be calculated using a program available online at The Bioinformatics Center at Rensselaer and Wadsworth website (bioinfo.rpi.edu).

One manner of expressing hybridization potential is as ΔG (change in Gibbs free energy) in units of kcals/mol. In an embodiment, candidate sequences having hybridization potential with any naturally occurring biological sequence of a magnitude greater than or equal to −5 kcal/mol are discarded. In an embodiment, candidate sequences having hybridization potential with any naturally occurring biological sequence of a magnitude greater than or equal to −10 kcal/mol are discarded. In an embodiment, candidate sequences having hybridization potential with any naturally occurring biological sequence of a magnitude greater than or equal to −15 kcal/mol are discarded.

In some embodiments, the selected biological probes from the array of interest and/or the pool of candidate probes are screened by their predicted melting temperature with their respective hypothetical complements. In the denaturation of DNA, melting temperature is taken as the midpoint of the helix-to-coil transition. It will be appreciated that there are many different algorithms known to those of skill in the art that allow the prediction of melting temperature based on primary structure (the sequence itself). Examples of such algorithms include that described in Dimitrov and Zuker, 2004, Biophysical Journal, 87:215-226. The higher the melting temperature, the more energetically stable the duplex or hybridization is.

In an embodiment, candidate sequences having a predicted melting temperature outside the range of about 75° C. to about 85° C., assuming molecule concentrations of between about 1×10⁻⁸ M and 1×10⁻¹⁰ M, are discarded. In an embodiment, candidate sequences having a predicted melting temperature outside the range of about 78° C. to about 82° C., assuming molecule concentrations of between about 1×10⁻⁸ M and 1×10⁻¹⁰ M, are discarded. In an embodiment, candidate sequences having a predicted melting temperature outside the range of about 79.5° C. to about 80.5° C., assuming molecule concentrations of between about 1×10⁻⁸ M and 1×10⁻¹⁰ M, are discarded.

Thermodynamic properties related to the formation of stable structures, such as hairpins, can be calculated in an analogous manner to those of duplex formation. This information can similarly be used to reject candidate sequences if it is likely that the probe will exist in a hairpin formation in solution under the hybridization conditions.

Some embodiments include screening techniques that rely on dataset(s) containing known biological sequences from the organism of interest. Some arrays are designed for use with samples taken from specific organisms. The specific organism(s) that a given array is designed to test samples from is the “organism(s) of interest”. Many projects being conducted by those of skill in the art continue to add to the total pool of known biological sequences for many different organisms. The dataset used for similarity screening can be drawn from one or more databases.

Exemplary databases containing known biological sequences include the NCBI nt database (ncbi.nih.gov), the TIGR (The Institute for Genomic Research) gene indices (tigr.org/tdb/tgi/index.shtml), and the NCBI's Unigene datasets (ncbi.nlm.nih.gov/entrez/query.fcgi?db=unigene). In some embodiments, screening techniques are performed against one or more of the NCBI nt dataset, the TIGR gene indices, and the NCBI's Unigene unique datasets for H. sapiens, A thaliana, and C. elegans.

Those of skill in the art will appreciate that there are also other databases that are available and that contain additional sequences from many different organisms. Publicly available sequence databases include those maintained by: GenBank (Bethesda, Md. USA) (ncbi.nih.gov/genbank/), European Molecular Biology Laboratory's European Bioinformatics Institute (EMBL-Bank in Hinxton, UK) (ebi.ac.uk/embl/), the DNA Data Bank of Japan (Mishima, Japan) (ddbj.nig.ac.jp/), the Ensembl project (ensembl.org/index.html), and The Institute for Genomic Research (TIGR) (tigr.org). Examples of databases that can be obtained and/or searched through the NCBI web portal (ncbi.nih.gov) include Entrez Nucleotides (including data from GenBank, RefSeq, and PDB), all divisions of GenBank, RefSeq (nucleotides), dbEST, dbGSS, dbMHC, dbSNP, dbSTS, TPA, UniSTS, PopSet, UniVec, WGS, Entrez Protein (including data from SwissProt, PIR, PRF, PDB, and translations from annotated coding regions in GenBank and RefSeq), RefSeq (proteins), and many others.

It will be appreciated that some datasets are directed to certain types of sequence information. By way of example, some datasets are directed to genomic sequences, while other datasets are directed to expressed sequences. Still other datasets are directed to polypeptide sequences. The appropriate dataset for use will depend on both the type of array intended (CGH, expression, etc.) and the identity of the organism of interest.

Some embodiments include using a computer system to screen candidate sequences against databases of known sequences. Many available sequence databases can be accessed with computer programs in a way that facilitates automated screening of candidate sequences. Some embodiments include a computer program that automatically screens candidate sequences against databases of known sequences.

Some embodiments include empirically validating candidate sequences. Candidate sequences can be empirically validated by putting the sequences on a test array and then testing hybridization of a sample with sequences on the test array. In the example of CGH arrays, the test sample used for validation testing can simply include any type of DNA containing sample from the organism since any normal diploid cell line contains the entire genome of the organism. In the example of expression arrays, since no single RNA sample includes all targets that can be expressed in any cell, the test sample will frequently represent a variety of tissue types or tissue conditions. By way of example, the test sample can include a mixed tissue sample (such as Universal Reference RNA, available from Stratagene, La Jolla, Calif.), a highly expressive cell line (such as HeLa), and/or a collection of tissues including unusual tissue types such as stressed cells, fetal tissue, and the like. In an embodiment, candidate sequence testing includes demonstrating that DNA and/or RNA from the test sample does not hybridize to the control probes under conditions (e.g., temperature, salt concentrations, sample concentrations, etc.) similar to that expected to be used in the hybridization protocol for the array of interest.

With arrays that are read by detecting fluorescence, the substrate may be of a material that emits low fluorescence upon illumination with the excitation light. Additionally, the substrate may be relatively transparent to reduce the absorption of the incident illuminating laser light and subsequent heating if the focused laser beam travels too slowly over a region.

Thus, as discussed above, aspects of the invention are described further below.

In one aspect, the invention provides a method for screening candidate probe sequences sequence comprising: selecting a subset of probe sequences from a set of sequences randomly; generating a plurality of candidate probe sequences by randomly permuting the selected probe sequence; and screening the candidate probe sequences for sequence similarity to biologically occurring sequences. In another aspect, the method further comprises selecting a negative probe sequence from the candidate probe sequences wherein the negative probe sequence does not have significant sequence similarity to the biologically occurring sequences.

Probe sequences can additionally be screened based on melting temperature (Tm). In one aspect, the method comprises discarding candidate sequences having a melting temperate (Tm) outside the range of about 78° C. to about 82° C.

In certain aspects, one or more steps of the method may be performed using a computer.

In certain aspects, the biologically occurring sequences comprise at least 50%, at least 90% or the entire genome of a biological organism, for example, the genome of a mammal such as a human being. In other aspects, the biologically occurring sequences comprise at least 50%, at least 90% or the entire transcriptome of a biological organism, for example, the transcriptome of a mammal such as a human being. In one aspect, screening the candidate probe sequences for sequence similarity to biologically occurring sequences comprises screening a set of candidate probe sequences against a database of known sequences. In another aspect, the set of sequences includes sequences complementary to nucleic acid sequences from an organism of interest, and the database comprises sequences from the organism of interest.

In certain aspects, screening the candidate probe sequences for sequence similarity to biologically occurring sequences comprises subdividing each candidate probe sequence into a plurality of corresponding candidate probe subsequences. The method can further comprise scoring the sequence similarity of each candidate probe sequence according to the sequence similarity of the corresponding candidate probe subsequences.

A method according to an aspect of the invention can further comprise generating a database of negative probe sequences. As discussed above, in certain aspects, a negative probe sequence does not have significant sequence similarity to biologically occurring sequences, such as for example, the genomic sequences of an organism (e.g., a mammal, such as a human being). In certain aspects, the genomic sequences comprise at least about 50%, at least 90% or 100% of the genomic sequences of an organism, such as a mammal (e.g., a human being). In certain other aspects, the biologically occurring sequences comprise the sequences of a transcriptome and in further aspects, at least 50%, at least 90%, or 100% of the transcriptome of a mammal, such as a human being.

In one aspect, the method comprises receiving sequence information for a negative probe sequence and synthesizing the negative probe sequence. Probe sequences can be synthesized by a variety of methods, including, but not limited to in situ synthesis on a solid support (e.g., an array substrate).

The method may further include empirically testing candidate probe sequences by contacting the probe sequences to a test sample of target sequences and monitoring binding of the probe sequences to the target sequences. For example, candidate probe sequences can be included on an array substrate which can then be contacted with target sequences. The array substrate can additionally include one or more test sequences designed to specifically hybridize to one or more sequences in a biological sample comprising the biologically occurring sequences. In a further aspect, the array substrate includes a positive control probe comprising a sequence known to be complementary to a sequence in the sample or a sequence which is spiked into the sample.

A negative probe sequence can be included in a probe set, which can be immobilized on an array, in certain aspects, for a variety of hybridization-based assays. For example, the probe sequence can be included on an array used in a CGH assay, a location analysis assay, a gene expression assay and the like. Optionally, the probe can be empirically validated as described above before inclusion in the probe set. In one aspect, a negative probe sequence comprises a sequence selected from SEQ ID. NOS. 1-9. Additional aspects of the invention include a probe set comprising at least two nucleic acid molecules comprising sequences selected from the group consisting of SEQ ID. NOS. 1-9 and an array comprising one or more probe sequences selected from the group consisting of SEQ ID NOS. 1-9.

In one aspect, a method according to the invention further comprises synthesizing one or more negative control probe sequences. In another aspect, a negative control probe sequence comprises a sequence length of 10 to 200 bases. In another aspect, a negative control probe sequence comprises a sequence length of 60 bases. In certain aspects according to the invention, a probe includes a negative control sequence and a cleavable site for releasing the negative control probe from an array substrate on which it is immobilized. The probe can additionally or optionally include primer recognition sites for binding to a primer so that the probe can be copied in the presence of a primer, a polymerase and suitable reagents for performing a primer extension and/or amplification reaction.

In another aspect, the invention further provides a probe sequence comprising a negative control probe sequence and a biological probe sequence (i.e., a sequence designed to specifically hybridize to a biologically occurring sequence) for detecting a target sequence in a sample. In one aspect, the negative control probe sequence is proximal to a solid support on which the probe is immobilized, to link the biological probe sequence to the solid support (either directly or via an additional chemical moiety to which the negative control probe sequence is attached). In a further aspect, an additional parameter used to screen the negative control probe sequence is an absence of secondary structure or ability to form hairpins, such that the negative control probe sequence has minimal likelihood of forming secondary structure. In certain aspects, the negative control probe sequence moves the biological probe sequence off the surface of the microarray and increases hybridization potential of the biological probe sequence (e.g., by reducing steric hindrance and increasing overall sequence accessibility).

In another aspect of the invention, the invention provides an array comprising at least one probe comprising a negative control probe sequence and a biological probe sequence. In still another aspect, the array comprises a plurality of probes comprising a negative control probe sequence and a biological probe sequence. Within the plurality, the negative control probe sequences can be the same or different in one aspect, though in another aspect, they are the same. Similarly, within the plurality the biological probe sequence can be the same or different, though in one aspect, the biological probe sequences are different. In still other aspects, the plurality can comprise the same negative control probe sequences and different biological probe sequences.

In one aspect, the invention also provides a computer readable medium having computer-executable instructions for performing steps of methods as described herein.

In another aspect, the invention provides an apparatus for screening candidate probe sequences, the apparatus comprising: a memory store; and a programmable circuit in electrical communication with the memory store, the programmable circuit programmed to select probe sequences from a set of sequences randomly; generate a plurality of candidate probe sequences by randomly permuting the selected biological probe sequence; and to screen the candidate probe sequences for sequence similarity to biologically occurring sequences. The circuit can be further programmed to select a probe sequence from the candidate probe sequences that does not have significant sequence similarity to the biologically occurring sequences. The programmable circuit can be further programmed to screen candidate probe sequences other properties, such as melting temperature (Tm), for example. In one aspect, the apparatus further comprises or communicates with a nucleic acid synthesis device, such as an inkjet printer for printing a nucleic acid array. In another aspect, the nucleic acid synthesis device is responsive to the programmable circuit (e.g., directly or indirectly).

In a further aspect, the invention provides a system comprising a database of negative control probe sequences. In one aspect, sets of negative control probe sequences are selected which correspond to sets of different biologically occurring sequences. A set includes a least one collection of nucleic acid sequences for a biological sample of interest—for example, the set may include human genomic sequences for a biological sample from a human being. In certain aspects, the set includes a plurality of different collections of biologically occurring sequences. For example, a set can comprise mouse genomic sequences and human genomic sequences, such that the database includes a set of negative control probes for a sample of mouse genomic sequences and a set of negative control probes for a sample of human genomic sequences. In one aspect, the system further comprises a search engine for searching the database in response to an input identifying a set of biologically occurring sequences. For example, in one aspect, in response to a user request for negative control probes for a sample of human genomic nucleic acids, the search engine will search the database to identify those negative control probe sequences that do not have significant similarity to any human genomic sequences.

In certain aspects, the system communicates with a user device comprising a display for displaying data relating to the negative probe sequences. The data can include but is not limited to: annotation data, sequence data, data relating to empirically determined hybridization properties of the probes, etc. In a further aspect, in response to a selection of one or more negative control probes (e.g., by selecting appropriate areas on a graphical user interface or display), a user can communicate an order for the one or more negative control probes to an entity that can provide the user with such probes (e.g., synthesized on an array or provided in a lyophilized form or in solution).

### EXAMPLE

Embodiments may be better understood with reference to the following example. This example is intended to be representative of specific embodiments but is not intended as limiting the scope.

### Example 1

### Generation of Negative Control Sequences

While it will be appreciated that there are many different techniques for implementing embodiments as program code, this example provides a Matlab script as a specific example. The script takes biological probe sequences and creates random permutations of the sequences to generate a pool of random candidate sequences. The script then subdivides the candidate sequences into subsequences and checks for significant sequence similarity against a table containing known sequences from an organism of interest. The script then creates histograms for similarity scoring purposes.

```
%MAKENEGATIVECONTROLPROBES (Matlab script)
Multiplier=20;
%Biological Probe Sequences:
lod Sequences.mat
for i=1:Multiplier
  %The scramble function randomly permutes the sequences:
  ScrambleSeqs=scramble(Sequences);
    if i==1
      Table60mers.Sequence=ScrambleSeqs;
else
  Table60mers.Sequence=[Table60mers.Sequence;ScrambleSeqs];
        end
end
Table60mers.ProbeID=[1:length(Table60mers.Sequence)]’;
Table60mers.Start=ones(size(Table60mers.ProbeID));
%Tile 30-mer sub-probes through 60-mer probes at15-base intervals:
Table30mers=subdivideprobes(Table60mers,30,15);
Table30mers.ProbeID60mer=Table30mers.ProbeID;
Table30mers.ProbeID=
Table30mers.ProbeID*1000+Table30mers.Start;
save WGA2_CandNegCont_Set2_Table30mers.mat Table30mers
save WGA2_CandNegCont_Set2_Table60mers.mat Table60mers
List30.ProbeID=Table30mers.ProbeID;
List30.Sequence=Table30mers.Sequence;
%export a text file that can be used by ProbeSpec for homology
search of 30-mer test-sequences against human genome:
table2tabtext(List30,
‘WGA2_CandNegCont_Set2_Table30mers.1st’)
%RUN PROBESPEC
% load the resulting homology search file with a histogram of hits at
various distances from 0-9 bases from the original 30-mer sequences:
% load HomologyTable:
load WGA2_CandNegCont_Set2_30mers_MAP.mat
% load Table30mers:
load WGA2_CandNegCont_Set2_Table30mers.mat
% join Table30mers & HomologyTable on ProbeID:
HomologyTable.ProbeID=double(HomologyTable.ProbeID)
NewTable30mers=tablejoin(‘left’,Table30mers,HomologyTable,
‘ProbeID’,‘=’, ‘ProbeID’)
load WGA2_CandNegCont_Set2_Table60mers.mat
% combine 30mer probes to make 60mer probes:
% add histogram information for each triplet of 30-mer subsequences:
HomologyTable60mers=
combinesubseqhomologies(NewTable30mers,
‘ProbeID60mer’,‘Start’)
NewTable60mers=
tablejoin(‘left’,Table60mers,HomologyTable60mers,‘ProbeID’,‘=’,
‘UniFullSeqID’)
% Score homologies for each probe, generate HomLogS2B score:
[HomLogS2B,HomCat,NewTable60mers]=
categorizehomology(NewTable60 mers,1);
save NC_60mersHomologyTable.mat NewTable60mers
```

% Keep only those probes with the best homology scores,
HomLogS2B.
figure, %plot resulting homology score distribution:
hist(Table.HomLogS2B,
[floor(min(Table.HomLogS2B)):ceil(max(Table.Hom LogS2B))])

The probes listed below have been empirically validated as showing little hybridization ability.

SEQ ID NO. 1:
5′-TATCCTACTATACGTATCACATAGCGTTCCGTATGTGGCCGGGATAGACCTAGCTTAAGC-
3′
SEQ ID NO. 2:
5′-
ACTCAAATACGGCCGATCTCCGTAGTAAGGCATCCAACCTGCGATACTAGCCACTTCCCG-3′
SEQ ID NO. 3:
5′-
ACAGCCAACTAATCCGGGATACCGCCGTTATTCGACTAATCCCGGGACGTCAAGTTCCAC-3′
SEQ ID NO. 4:
5′-
CCGCGCGGCATGAAGTATGCAGCGCTCGAGCCTAGTCATTCGTAAGCGATATGTTTAGTG-3′
SEQ ID NO. 5:
5′-CGTTTCTACGCGTACGCCTTTATGTCGAGGCAACGCCTCGGTGTACTCCTACGGGTTTTG-
3′
SEQ ID NO. 6:
5′-
ACTGATTGCCGTGTATTAGCCGGTCGGTAACTCGGTTCCGCTACTAGCGCGCCAGATTTC-3′
SEQ ID NO. 7:
5′-
CTAACGGGTCCAAGACGCGCAACATTATGTAGCGTACTAGGACCCTAACTGCGACTATCC-3′
SEQ ID NO. 8:
5′-
CCATAAGGCGGACCCAGATCGATTGACGGGTGGCTAGATATGTCGTGCTTAGTTCCCAAA-3′
and
SEQ ID NO. 9:
5′-
AGTATGTGTAGCGAGGAGCTAGTCGTCGGTGCACAATCGGCCTAGAATTAGTTGCCTCGA-3′

The various embodiments described above are provided by way of illustration only and should not be construed to limit the claims. Those skilled in the art will readily recognize various modifications and changes that may be made without following the example embodiments and applications illustrated and described herein, and without departing from the true spirit and scope of the disclosure or the following claims.

What is claimed is:

1. A method for screening candidate probe sequences for use as negative probe sequences in a hybridization assay, said method comprising:
randomly selecting a subset of probe sequences from a set of sequences that includes sequences complementary to nucleic acid sequences from an organism of interest;
generating a plurality of candidate probe sequences by randomly permuting the selected probe sequences;
screening the candidate probe sequences for sequence similarity to biologically occurring sequences to identify negative probe sequences; and
recording said negative probe sequences to a computer-readable medium.

2. The method of claim 1, wherein the method further comprises selecting a negative probe sequence from the candidate probe sequences wherein the negative probe sequence does not have significant sequence similarity to the biologically occurring sequences.

3. The method of claim 1, further comprising screening said candidate probe sequences based on GC content range

4. The method of claim 2, comprising discarding candidate probe sequences having a GC content range outside of 0.36-0.39.

5. The method of claim 1, further comprising screening said candidate probe sequences based on melting temperature (Tm).

6. The method of claim 4, comprising discarding said candidate probe sequences having a melting temperate (Tm) outside the range of about 78° C. to about 82° C.

7. The method of claim 1, wherein screening the candidate probe sequences for sequence similarity to biologically occurring sequences comprises screening the candidate probe sequences against a database of known sequences.

8. The method of claim 1, wherein said set of sequences are in a database comprising sequences from said organism of interest.

9. The method of claim 1, wherein screening the candidate probe sequences for sequence similarity to biologically occurring sequences comprises subdividing each candidate probe sequence into a plurality of corresponding candidate probe subsequences.

10. The method of claim 9, further comprising scoring the sequence similarity of each candidate probe sequence according to the sequence similarity of the corresponding candidate probe subsequences.

11. The method of claim 1, further comprising empirically testing said candidate probe sequences by contacting said candidate probe sequences to a test sample of target sequences and monitoring binding of said candidate probe sequences to said target sequences.

12. The method of claim 11, wherein the candidate probe sequences are included on an array substrate.

13. The method of claim 12, wherein empirically testing the candidate probe sequences on the array comprises hybridizing the array with the test sample.

14. The method of claim 12, wherein the array is used in a CGH assay.

15. The method of claim 12, wherein the array is used in a location analysis assay.

16. The method of claim 12, wherein the array is used in a gene expression assay.

17. The method of claim 12, wherein the array is suitable for performing an expression assay.

18. The method of claim 1, further comprising synthesizing a plurality of negative control probe sequences.

19. The method of claim 2, wherein the negative control probe sequences have a sequence length of 10 to 200 bases.

20. The method of claim 2, wherein the negative control probe sequences have a sequence length of 60 bases.

21. The method of claim 1, wherein at least some of the steps are performed by a computer system.

22. A computer-readable medium having computer-executable instructions for performing the steps recited in claim 1 and reporting the results to a user.

23. An apparatus for screening candidate probe sequences, the apparatus comprising:
a memory store; and
a programmable circuit in electrical communication with the memory store, the programmable circuit programmed to
select probe sequences from a set of sequences randomly to produce selected probe sequences;
generate a plurality of candidate probe sequences by randomly permuting said selected probe sequences to produce candidate probe sequences;
screen said candidate probe sequences for sequence similarity to biologically occurring sequences to identify negative probe sequences; and
report the result to a user of said apparatus.

24. The apparatus of claim 23, wherein the circuit is further programmed to select a probe sequence from the candidate probe sequences that does not have significant sequence similarity to the biologically occurring sequences.

25. The apparatus of claim 23, the programmable circuit further programmed to screen the candidate probe sequences based on melting temperature (Tm).

26. The apparatus of claim 23, further comprising or communicating with an array printer, the array printer responsive to the programmable circuit.

27. An isolated nucleic acid molecule comprising a sequence selected from the group consisting of: SEQ ID NO. 1, SEQ ID NO. 2. SEQ ID NO. 3, SEQ ID NO. 4, SEQ ID NO. 5, SEQ ID NO. 6, SEQ ID NO. 7, SEQ ID NO. 8, and SEQ ID NO. 9.

28. An array comprising one or more probe sequences selected from the group consisting of SEQ ID NOS.1

29. A probe set comprising at least two nucleic acid molecules comprising sequences selected from the group consisting of SEQ ID NO. 1, SEQ ID NO 2, SEQ ID NO. 3, SEQ ID NO. 4, SEQ ID NO. 5, SEQ ID NO. 6, SEQ ID NO. 7, SEQ ID NO 8, and SEQ ID NO. 9.

30. The method of claim 2, further comprising generating a database of negative probe sequences.

31. The method of claim 30, wherein the biologically occurring sequences comprise known genomic sequences.

32. The method of claim 31, wherein the genomic sequences comprise at least about 50% of the genomic sequences of a mammal.

33. The method of claim 31, wherein the genomic sequences comprise at least about 90% of the genomic sequences of a mammal.

34. The method of claim 32, wherein the mammal is a human being.

35. The method of claim 30, wherein the biologically occurring sequences comprise the sequences of a transcriptome.

36. The method of claim 35, wherein the transcriptome sequences comprise at least about 50% of the transcriptome of a mammal.

37. The method of claim 35, wherein the transcriptome sequences comprise at least about 90% of the transcriptome of a mammal.

38. The method of claim 37, wherein the mammal is a human being.

39. A method comprising receiving sequence information for a negative probe sequence designed according to the method of claim 2, and synthesizing the negative probe sequence.

40. The method of claim 39, wherein the negative probe sequence is synthesized in situ on an array substrate.

41. A system comprising a database of negative probe sequences selected according to the method of claim 2 for a set of biologically occurring sequences, and a search engine for searching the database in response to an input identifying a collection of biologically occurring sequences within the set.

42. The system of claim 41, wherein the system communicates with a user device comprising a display for displaying data relating to the negative probe sequences.

43. The system of claim 42, wherein the data comprises annotation data.

44. The system of claim 42, wherein the data comprises sequence data.

45. The system of claim 42, wherein the data comprises empirical data relating to hybridization properties of the probe to a test sample comprising a set of biologically occurring sequences.

46. An isolated polynucleotide probe comprising a negative control probe sequence selected according to the method of claim 2 and a biological probe sequence.

47. A set of isolated polynucleotide probes comprising at least two probes according to claim 46, wherein the biological probe sequences of each probe is different and the negative control probe sequence Is the same or different.

48. An array comprising a polynucleotide probe according to claim 46.

49. The method according to claim 1, further comprising synthesizing one or more of said negative probe sequences.


SEQUENCE LISTING (9 sequences)

SEQ ID NO: 1 - DNA, 60 bp, human
tatcctactatacgtatcacatagcgttccgtatgtggccgggatagacctagcttaagc

SEQ ID NO: 2 - DNA, 60 bp, human
actcaaatacggccgatctccgtagtaaggcatccaacctgcgatactagccacttcccg

SEQ ID NO: 3 - DNA, 60 bp, human
acagccaactaatccgggataccgccgttattcgactaatcccgggacgtcaagttccac

SEQ ID NO: 4 - DNA, 60 bp, human
ccgcgcggcatgaagtatgcagcgctcgagcctagtcattcgtaagcgatatgtttagtg

SEQ ID NO: 5 - DNA, 60 bp, human
cgtttctacgcgtacgcctttatgtcgaggcaacgcctcggtgtactcctacgggttttg

SEQ ID NO: 6 - DNA, 60 bp, human
actgattgccgtgtattagccggtcggtaactcggttccgctactagcgcgccagatttc

SEQ ID NO: 7 - DNA, 60 bp, human
ctaacgggtccaagacgcgcaacattatgtagcgtactaggaccctaactgcgactatcc

SEQ ID NO: 8 - DNA, 60 bp, human
ccataaggcggacccagatcgattgacgggtggctagatatgtcgtgcttagttcccaaa

SEQ ID NO: 9 - DNA, 60 bp, human
agtatgtgtagcgaggagctagtcgtcggtgcacaatcggcctagaattagttgcctcga